(19) United States
(12) Patent Application Publication
(10) Pub. No.: US 2017/0281209 A1
(43) Pub. Date: Oct. 5, 2017

(54) TONGUE CLEANER

(71) Applicant: The Procter & Gamble Company, Cincinnati, OH (US)

(72) Inventors: Nayda Liz RAMOS-MEDINA, Cincinnati, OH (US); Philip Andrew SAWIN, Cincinnati, OH (US)

(21) Appl. No.: 15/476,658

(22) Filed: Mar. 31, 2017

Related U.S. Application Data
(60) Provisional application No. 62/317,012, filed on Apr. 1, 2016.

(51) Int. Cl. A61B 17/24; A46B 15/00; A61C 17/22; A46B 9/04

(52) U.S. Cl. CPC A61B 17/244; A46B 9/04; A46B 15/0081; A61C 17/222

(57) ABSTRACT

A tongue cleaner comprises a base and a plurality of projections outwardly extending therefrom and including at least first projections and second projections. At least some of the projections have a first end attached to the base, a second free end opposite to the first end, and include an elongated stem and a head at the second end. The head has at least one cantilevered portion outwardly extending from the stem and having a bottom surface generally facing the base. A substantial portion of the bottom surface of the individual first projection is inclined away from the stem; and a substantial portion of the bottom surface of the individual second projection is inclined towards the stem.

### FIELD OF THE INVENTION

The present invention is in the field of oral care, and more specifically oral physiotherapy. The invention concerns a tongue-cleaning device comprising a plurality of flexible projections having unique configurations. The device is designed to remove from the tongue bacterial microorganisms, extraneous cellular debris, and the like, implicated in plaque formation on dental surfaces.

### BACKGROUND OF THE INVENTION

According to the American Dental Association, a major source of bad breath in healthy people is microbial deposits on the tongue, where a bacterial coating harbors organisms and debris that contribute to bad breath. Tissue in the mouth, and especially the tongue, is a “haven” for the growth of microorganisms. The dorsum of the tongue comprises papillae. The anterior two-thirds of the dorsum are covered with short fungiform papillae while the posterior third is covered with fungiform papillae interspersed with 2-3 mm long filiform papillae. The dorsum of the tongue is one of the main foci of microorganisms in the oral cavity; many bacterial species resident on the tongue colonize and accumulate in plaque, and these bacteria are normally integral to dental calculus.

The papillary nature of the tongue surface creates a unique ecological site that provides an extremely large surface area, favoring the accumulation of oral bacteria. Because the tongue is a site of dense bacterial populations and because plaque forms relatively fast after its removal, the tongue can be a source of dental plaque. Bacteria, food particles and the breakdown products of foods are trapped in the crevices between the papillae, particularly on the posterior portion of the tongue's dorsum. This material is the source of most oral bacteria and odor.

The tongue, besides harboring large populations of microorganisms, also continuously sheds cells of the surface layer of epithelium. The availability of oral debris from these sources could contribute to plaque formation on dental surfaces. In addition, streptococcus salivarius and related species can be found on the dorsum of the tongue. Anaerobic flora and bacteria residing on the tongue and other soft tissues in the mouth play an important role in the development of chronic bad breath commonly called halitosis. In general, the bacteria produce volatile sulfur compounds (VSC). Buildup of those sulfur compounds on the tongue's surface can lead to bad breath or oral malodor.

Known tongue scrapers have generally not been too effective. Further, many of these scrapers cannot easily be used to cleanse other tissue within the mouth. Notwithstanding the benefits to be gained by cleaning the tissue in the mouth, many users avoid the use of such cleansers due to a lack of comfort and/or ease of use. The present description seeks to overcome at least some of these limitations and to provide new features heretofore not available.

### SUMMARY OF THE DISCLOSURE

The present disclosure is directed to a tongue cleaner comprising a base and a plurality of projections outwardly extending from the base. The base can be rigid or flexible. The base has a first direction and a second direction perpendicular to the first direction. In one embodiment, the first direction is a machine direction (MD) and the second direction is a cross-machine direction (CD). One skilled in the art will readily understand that the MD and CD can be mutually reversed. Further, the terms “MD” and “CD” may be used herein interchangeably with terms “first direction” and “second direction.”

The plurality of projections extends, preferably in a predetermined pattern, from the base in a third direction. The third direction can be generally normal to the base. At least one of the projections has a first end attached to the base, a second free end opposite to the first end, and a height extending between the first and second ends.

The individual projection comprises an elongated stem and a head, which is preferably made integral with the stem. The head is disposed at a distance from the base. The stem has a longitudinal axis oriented substantially parallel to the third direction.

The head comprises at least a first cantilevered portion and a second cantilevered portion, both outwardly extending from the stem. The first and second cantilevered portions can be mutually opposite. The cantilevered portions outwardly extend from the stem in directions that can be substantially perpendicular to the third direction or inclined relative to the third direction. The cantilevered portions can extend from the stem at an angle or angles relative to the base.

Each of the cantilevered portions has a bottom surface generally facing the base and a top surface opposite to the bottom surface. In one embodiment, the bottom surface and the top surface terminate by abutting one another to form, in combination with one another, an edge disposed at a distance from the stem.

In one aspect, the disclosure is directed to a tongue cleaner comprising a base having a first direction and a second direction perpendicular to the first direction and a plurality of projections outwardly extending from the base in a third direction, wherein at least one of the projections has a first end attached to the base, a second free end opposite to the first end, and a height extending between the first end and the second end, wherein the at least one of the projections comprises an elongated stem and a head integral with the stem and disposed at a distance from the base, the stem having a longitudinal axis oriented substantially parallel to the third direction, wherein the head comprises at least two mutually opposite cantilevered portions outwardly extending from the stem, each of the cantilevered portions having a bottom surface generally facing the base and a top surface opposite to the bottom surface, wherein a substantial portion of the bottom surface is inclined away from the stem.

In another aspect, the disclosure is directed to a tongue cleaner comprising a base having a first direction and a second direction perpendicular to the first direction and a plurality of projections outwardly extending from the base in a third direction, wherein at least one of the projections has a first end attached to the base, a second free end opposite to the first end, and a height extending between the first and second ends, wherein the at least one of the projections comprises an elongated stem and a head integral with the stem and disposed at a distance from the base, the stem having a longitudinal axis oriented substantially parallel to the third direction, wherein the head comprises at least two mutually opposite cantilevered portions outwardly extending from the stem, each of the cantilevered portions having a bottom surface generally facing the base and a top surface opposite to the bottom surface, wherein a substantial portion of the bottom surface is inclined towards the stem.

In still another aspect, the disclosure is directed to a tongue cleaner comprising a base having a first direction and a second direction perpendicular to the first direction and a plurality of projections outwardly extending from the base in a third direction, wherein at least some of the projections have a first end attached to the base, a second free end opposite to the first end, and a height extending between the first end and the second end, wherein the at least some of the projections comprise an elongated stem and a head integral with the stem and disposed at a distance from the base, the stem having a longitudinal axis, wherein the head comprises at least one cantilevered portion outwardly extending from the stem, the at least one cantilevered portion having a bottom surface generally facing the base and a top surface opposite to the bottom surface, wherein the plurality of projections comprises at least first projections and second projections, wherein a substantial portion of the bottom surface of the individual first projections is inclined away from the stem, and wherein a substantial portion of the bottom surface of the individual second projections is inclined towards the stem.

In one embodiment, the first projections and the second projections in combination form a pattern having a combined density of from about 60 projections per cm² to about 400 projections per cm² of the base. The combined density can be from about 75 projections per cm² to about 350 projections per cm² of the base.

In yet another aspect, the disclosure is directed to a tongue cleaner comprising a base having a first direction and a second direction perpendicular to the first direction; a plurality of projections outwardly extending from the base in a third direction, wherein at least one of the projections has a first end attached to the base, a second free end opposite to the first end, and a height extending between the first end and the second end, wherein the at least one of the projections comprises an elongated stem and a head integral with the stem and disposed at a distance from the base, the stem having a longitudinal axis oriented substantially parallel to the third direction, wherein the head comprises at least a first cantilevered portion outwardly extending from the stem and a second cantilevered portion outwardly extending from the stem, wherein the first cantilevered portion has a first bottom surface generally facing the base and a first top surface opposite to the first bottom surface, wherein the second cantilevered portion has a second bottom surface generally facing the base and a second top surface opposite to the first bottom surface, and wherein a substantial portion of the first bottom surface is inclined towards the stem while a substantial portion of the second bottom surface is inclined away from the stem.

In some embodiments of the tongue cleaner, at least one of the bottom surface and the top surface of the cantilevered portion can be at least partially curved. Such an entirely or partially curved bottom or top surface can have a shape selected from the group consisting of a convex shape, a concave shape, and any combination thereof. In some embodiments, the bottom surface or the top surface may comprise a portion or portions having a convex shape and a portion or portions having a concave shape, in combination.

The height of the individual projection can be from about 0.2 mm to about 0.55 mm. In one embodiment, the height of the individual projection can be from about 0.39 mm to about 0.47 mm. The first width of the head of the individual projection can be from about 0.2 mm to about 0.24 mm. The second width of the head of the individual projection can be from about 0.25 mm to about 0.6 mm. In one particular embodiment, the second width of the head is from about 0.34 mm to about 0.4 mm. The thickness of the head of the individual projection can be from about 0.02 mm to about 0.1 mm. In one particular embodiment, the thickness of the head is from about 0.03 mm to about 0.07 mm. The individual cantilevered portion may have a length of from about 0.07 mm to about 0.13 mm as measured from a top surface of the top section of the stem, wherein the top section's top portion is closest to the end of the cantilevered portion.

In plan view, the head of the tongue cleaner may comprise any suitable form or shape, e.g., a shape selected from a circular shape, a semi-circular shape, an elliptical shape, a polygon shape, a parallelogram shape, and any combination thereof.

The plurality of projections can be spaced from one another to form on the base a pattern having a first pitch extending parallel to the first direction and a second pitch extending parallel to the second direction. In one embodiment, this pattern comprises a staggered or alternating arrangement. The first pitch and the second pitch can be equal or otherwise. In one embodiment, the first pitch is greater than the second pitch. In one embodiment, the first direction is parallel to the machine direction (MD) and the second direction is parallel to the cross-machine direction (CD). In one particular embodiment, the first pitch is at least 50% greater than the second pitch, wherein the first pitch extends in a direction substantially perpendicular to the direction in which the first pitch extends. The first pitch can be from about 0.4 mm to about 1.4 mm, more specifically from about 0.8 mm to about 1.3 mm, and even more specifically from about 0.9 mm to about 1.2 mm. The second pitch can be from about 0.4 mm to about 0.7 mm, and more specifically from about 0.48 mm to about 0.62 mm.

The stem can have any suitable body. In one embodiment, the stem has a body comprising a top section adjacent to the head, a bottom section opposite to the top section, and a midsection between the top and bottom sections. One of the top section, the midsection, and the bottom section can have a tapering portion that is narrower than the midsection in its widest part. In one particular embodiment of the stem, the stem's midsection is wider, in at least one direction substantially perpendicular to the third direction, than the projection's head. In another embodiment, the head is wider than the bottom section of the stem as measured in a direction substantially perpendicular to the third direction. In still another embodiment, the bottom section of the stem is wider than the head as measured in a direction substantially perpendicular to the third direction.

The disclosure is also directed to an oral-care implement comprising the tongue cleaner as described herein, wherein the oral-care implement is selected from the group consisting of a manual toothbrush and an electric toothbrush.

### BRIEF DESCRIPTION OF THE DRAWINGS

FIG. 1 is a schematic axonometric view of an exemplary toothbrush having a tongue cleaner of the disclosure.

FIG. 2 is a schematic axonometric view of a portion of an exemplary embodiment of the tongue cleaner of the disclosure.

FIG. 2A is a schematic plan view of an exemplary embodiment of the tongue cleaner of the disclosure, comprising a pattern of aligned (non-staggered) projections outwardly extending from a base.

FIG. 2B is a schematic plan view of an exemplary embodiment of the tongue cleaner of the disclosure, comprising a pattern of staggered projections outwardly extending from a base.

FIG. 2C is a schematic plan view of an exemplary embodiment of the tongue cleaner of the disclosure, comprising a plurality of first projections and a plurality of second projections different from the first projections, wherein the plurality of the first projections and the plurality of the second projections are distributed on the base in a staggered arrangement relative to one another.

FIG. 3 is a schematic fragmental elevational cross-machine-directional side view of an exemplary embodiment of the tongue cleaner of the disclosure and showing a projection in which a substantial portion of the bottom surface of the cantilevered portions is inclined to face the stem.

FIG. 3A is a schematic fragmental elevational machine-directional side view of the exemplary embodiment of the tongue cleaner shown in FIG. 3.

FIG. 4 is a schematic fragmental elevational side view of another exemplary embodiment of the tongue cleaner of the disclosure and showing a projection in which a substantial portion of the bottom surface of the cantilevered portions is inclined to face the stem.

FIG. 5 is a schematic fragmental side view of an exemplary embodiment of the tongue cleaner of the disclosure and showing a projection in which a substantial portion of the bottom surface of the cantilevered portions is inclined to face away from the stem.

FIG. 6A is a top view of an exemplary embodiment of an individual projection comprising a head having a circular shape in plan view.

FIG. 6B is a top view of an exemplary embodiment of an individual projection comprising a head having an oval or elliptical circular shape in plan view.

FIG. 6C is a top view of an exemplary embodiment of an individual projection comprising a head having a rectangular shape in plan view.

FIG. 6D is a top view of an exemplary embodiment of an individual projection comprising a head having a shape comprising parallelogram in plan view.

FIG. 6E is a top view of an exemplary embodiment of an individual projection comprising a head having a shape comprising two mutually opposite straight sides and two mutually opposite curved sides.

FIG. 6F is a top view of an exemplary embodiment of an individual projection comprising a head having a polygonal shape in plan view.

FIG. 6G is a top view of an exemplary embodiment of an individual projection comprising a head having a rhombus shape in plan view.

FIG. 7 is a schematic fragmental side view of an exemplary embodiment of the tongue cleaner of the disclosure and showing a projection comprising a stem including a top section, a midsection, and a bottom section, wherein the midsection of the stem is wider than at least one of the head of the projection and the bottom portion of the stem in at least one of the first and second directions.

FIG. 8 is a schematic fragmental side view of an exemplary embodiment of the tongue cleaner of the disclosure and showing a projection comprising a stem including a top section and a bottom section, wherein the bottom section is wider than the head in at least one of the first and second directions.

FIG. 9 is a schematic plan view of an exemplary embodiment of the tongue cleaner of the disclosure, comprising a plurality of first projections and a plurality of second projections different from the first projections, wherein the plurality of the first projections and the plurality of the second projections are distributed on the base in a first direction and alternate relative to one another in a second direction.

FIG. 10 is a schematic plan view of an exemplary embodiment of the tongue cleaner of the disclosure, comprising a plurality of first projections and a plurality of second projections different from the first projections, wherein the plurality of the first projections and the plurality of the second projections are distributed on the base in an alternating arrangement relative to one another, wherein the first projections are bilaterally offset relative to the second projections.

FIG. 11 is a schematic plan view of an exemplary embodiment of the tongue cleaner of the disclosure, comprising a plurality of first projections and a plurality of second projections different from the first projections, wherein the plurality of the first projections and the plurality of the second projections are distributed on the base in an alternating arrangement relative to one another, wherein the first projections are unilaterally offset relative to the second projections.

FIG. 12 is a schematic plan view of an exemplary embodiment of the tongue cleaner of the disclosure, wherein a pattern of the projections on the base comprises a plurality of first units formed by first projections and a plurality of second units formed by second projections, bilaterally alternating.

FIG. 13 is a schematic plan view of another exemplary embodiment of the tongue cleaner of the disclosure, wherein a pattern of the projections on the base comprises a plurality of first units formed by first projections and a plurality of second units formed by second projections.

FIG. 14 is a schematic fragmental elevational side view of an exemplary embodiment of the tongue cleaner comprising a projection in which a head has a first cantilevered portion and a second cantilevered portion, wherein the first cantilevered portion has a first bottom surface and the second cantilevered portion has a second bottom surface, and wherein a substantial portion of the first bottom surface is inclined towards the stem while a substantial portion of the second bottom surface is inclined away from the stem.

FIG. 15 is a schematic axonometric view of a portion of an exemplary embodiment of the tongue cleaner similar to that shown in FIG. 2—but comprising projections that are inclined relative to the base.

FIG. 16 is a schematic fragmental elevational cross-machine-directional side view of an exemplary embodiment of the tongue cleaner shown in FIG. 15 and showing a projection in which a substantial portion of the bottom surface of the cantilevered portions is inclined to face the stem while the projection as a whole is inclined relative to the base.

FIG. 17 is a schematic fragmental elevational cross-machine-directional side view of an exemplary embodiment of the tongue cleaner showing a projection in which a stem is substantially perpendicular to the base while a head as a whole is inclined relative to the base.

FIG. 18 is a schematic fragmental elevational cross-machine-directional side view of an exemplary embodiment of the tongue showing a projection in which a stem is inclined relative to a base while a head as a whole is substantially parallel to the base.

### DETAILED DESCRIPTION

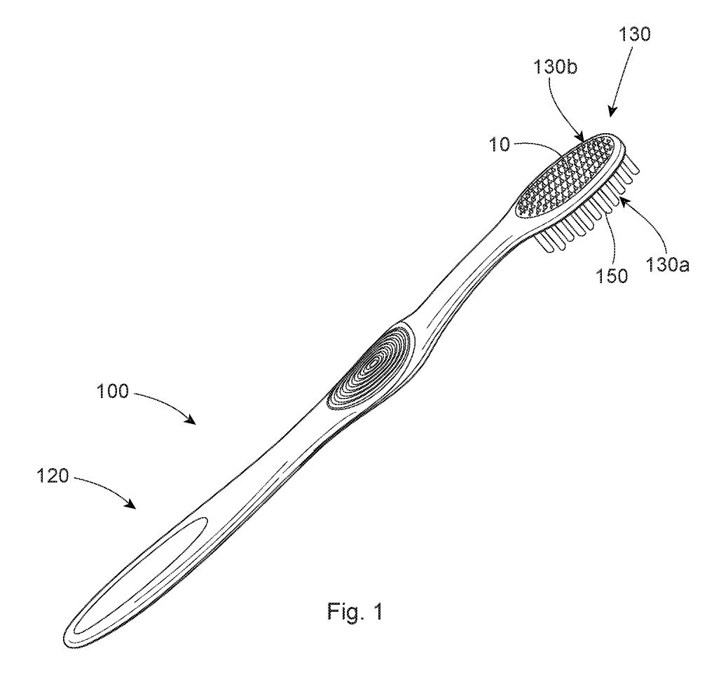

A tongue cleaner of the disclosure can be utilized in any suitable oral-care implement or device, such as, e.g., a manual toothbrush or an electric toothbrush—or may be structured as an independent, self-contained implement. In an exemplary embodiment of FIG. 1, a tongue cleaner 10 is part of a manual toothbrush 100 having a handle 120 and head 130 with a plurality of cleaning elements, or bristles, 150, extending from a first side 130a of the head 130. The tongue cleaner 10 can be conventionally disposed on the brush's head 130, more particularly on a second side 130b of the head 130, wherein the first and second sides 130a, 130b are mutually opposite. The tongue cleaner 10 could be constructed as an integral part of the body of the brush 100—and to comprise a material of the brush's body. Alternatively or additionally, the tongue cleaner 10 can be attached to the body of the brush 100 by any suitable means known in the art. In one embodiment, the tongue cleaner 10 can comprise a material of the cleaning elements 150 extending from the first side 130a of the head 130. In one particular embodiment, the tongue cleaner 10 can be made integral with at least some of the cleaning elements 150.

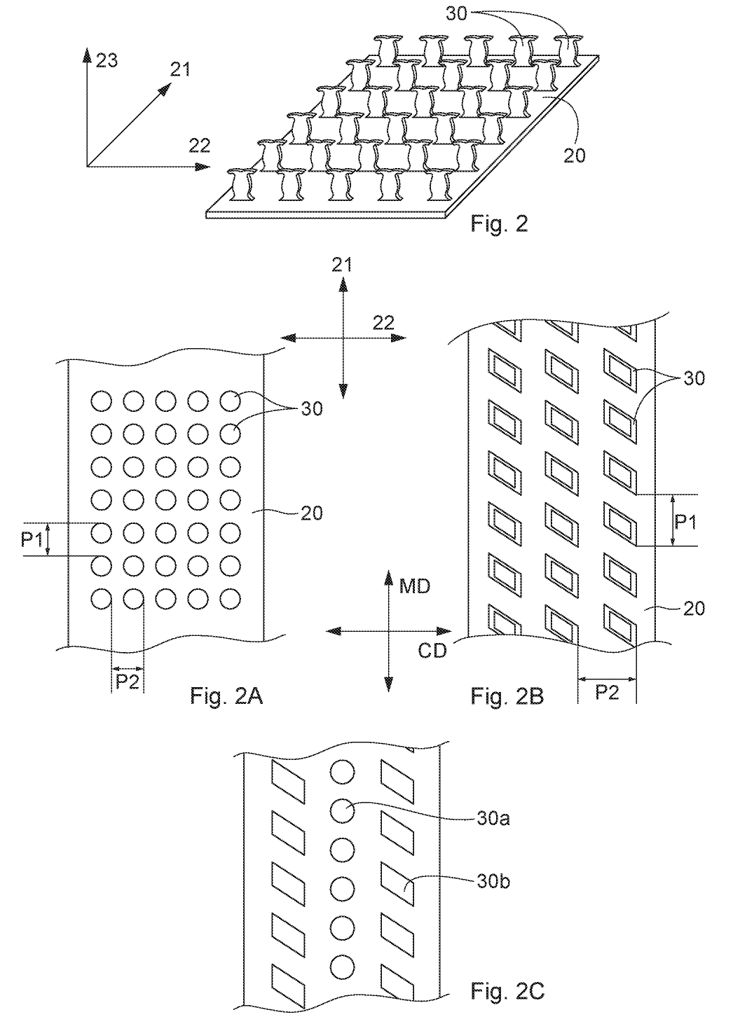

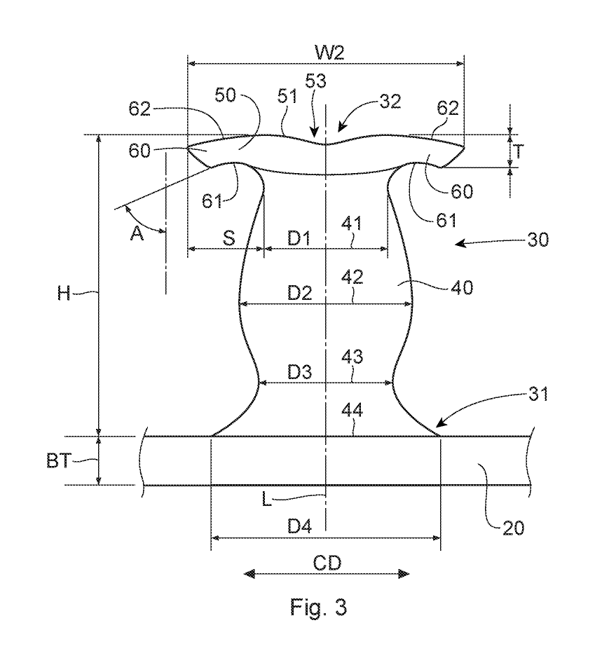

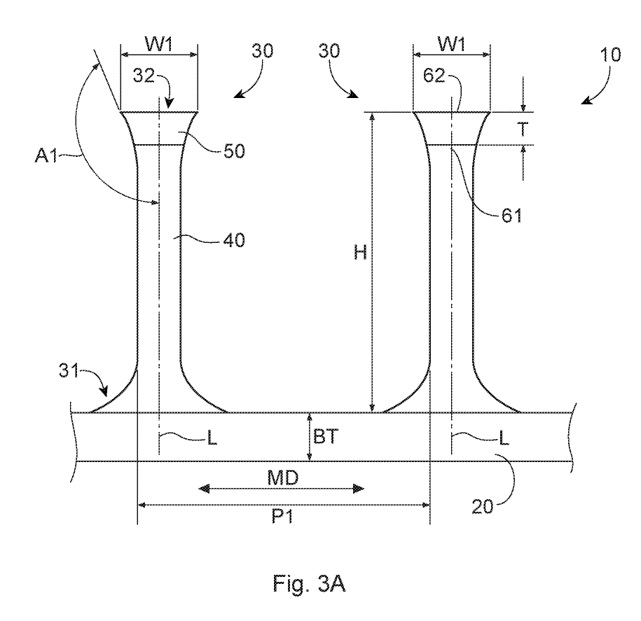

The tongue cleaner 10 comprises a base 20 and a plurality of projections 30 outwardly extending from the base 20, FIGS. 2, 2A, and 2B. The base can have any suitable thickness or “caliper” BT, FIGS. 3, 3A. The thickness BT of the base 20 can be, i.e., from 0.05 mm to 0.2 mm, or from 0.07 mm to 0.1 mm, or from 0.08 mm to 0.09 mm. The base 20 can be either rigid or flexible. The base 20 can be made of any suitable rigid or flexible material, including woven and non-woven materials. Also, the base 20 can comprises more than one layer of material. For example, the base 20 can comprise a first layer attached to a second layer, wherein the first layer comprises a first material and the second layers comprises a second material, and wherein the first material is different from the second material. The multi-layer embodiment of the base is not shown but would be easily visualized by a person skilled in the art.

The base 20 has a first direction 21 and a second direction 22 perpendicular to the first direction 21. In one embodiment, the first direction 21 is a machine direction (MD) and the second direction 22 is a cross-machine direction (CD). One skilled in the art will appreciate that the MD and CD directions can be conventionally reversed. The plurality of projections 30 extends, preferably in a predetermined pattern, from the base 20 in a third direction 23 different from the first direction 21 and the second direction 22.

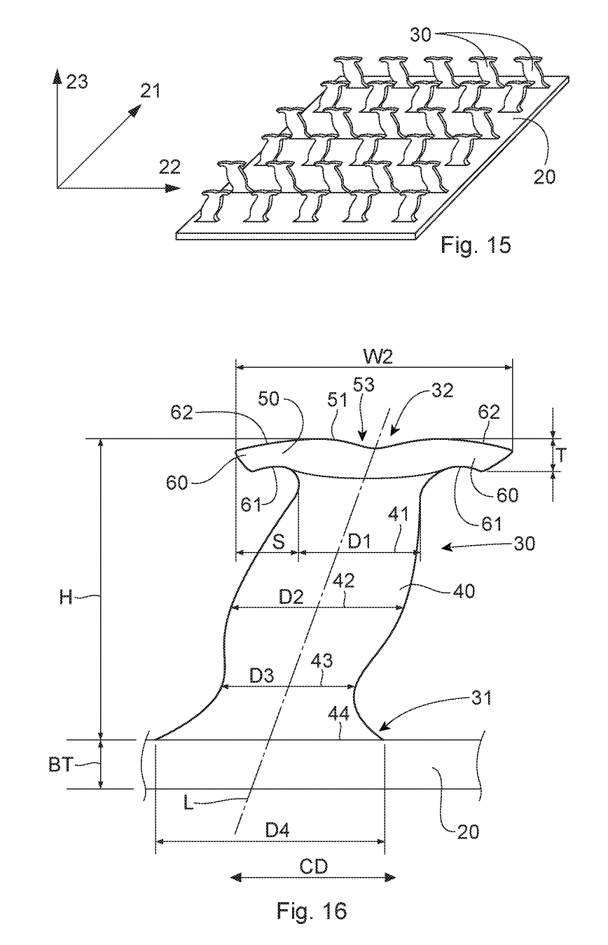

In some embodiments, the third direction 23 can be substantially perpendicular to the first direction 21 and the second direction 22—and hence substantially orthogonal to the base 20, FIGS. 2, 3. In other embodiments, the third direction 23 can be inclined relative to the base 20, so that the angle formed between the third direction 23 and the base 20 is greater or less than 90 degrees, FIGS. 15, 16. As used herein, the third direction 23 is defined as being generally normal to the first and second directions 21, 22. The term “generally normal” can be used herein, in the context of an angled orientation, to describe the orientation of the individual projections 30 outwardly extending from the base 20 in the third direction 23 that can be substantially orthogonal to the base 20 (and hence substantially perpendicular to the first and second directions 21, 22)—or inclined relative to the base 20 to comprise an acute or obtuse angle therewith not exceeding approximately 90±30 degrees. In other words, the term “generally normal” does not require strict or substantial perpendicularity between the third direction 23 and the base 20 (or between the third direction 23 and the first and second directions 21, 22). Instead, the term “generally normal” in the present context allows deviation of about±30 degrees from a strictly orthogonal orientation of the individual projection 30 relative to the base 20. Thus, the third direction 23 can comprise from about 60 degrees to about 120 degrees relative to the base 20.

As is shown in FIGS. 3, 4, and 5, the individual projection 30 has a first end 31, which is attached to, or integral with, the base 20, a second free end 32 opposite to the first end 31, and a height H extending between the first end 31 and the second end 32. The projection 30 comprises an elongated stem 40 terminating with a head 50 preferably integral with the stem 40 and disposed at a distance from the base 20. The stem 40 has a longitudinal axis L oriented substantially parallel to the third direction 23. Hence, the longitudinal stem L is generally normal to the base 20, i.e., the longitudinal axis L may be somewhat inclined (approximately 90 ±30 degrees) relative to the base 20, at an angle of from about 60 degrees to about 120 degrees relative to the base 20.

The head 50 has a first width W1, a second width W2, and a thickness T, FIG. 3A. The first width W1 can be oriented substantially in the first direction 21. This means that the first width W1 lies in a plane in which the first direction 21 does. In other words, for the first width W1 to be oriented substantially in the first direction 21, the first width W1 does not need to be parallel to the first direction 21; it is sufficient that the first width's cross-sectional projection taken in the plane of the first direction 21 be parallel to the first direction 21. The second width W2 can be oriented substantially in the second direction 22. Likewise, for the second width W2 to be oriented substantially in the second direction 22, the second width W2 does not need to be parallel to the second direction 22; it is sufficient that the second width's cross-sectional projection taken in the plane of the second direction 22 be parallel to the second direction 22. The second width W2 is be substantially perpendicular to the first width W1.

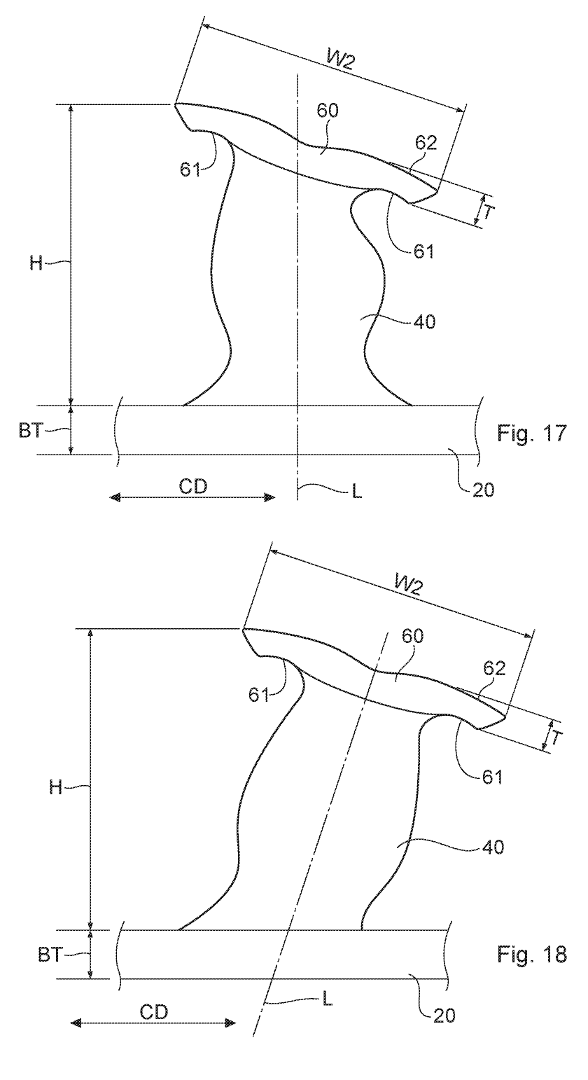

In some embodiments, the first width W1, the second width W2, or both the first and second widths W1, W2 may extend substantially perpendicular to the third direction 23. In some embodiments, the first width W1, the second width W2, or both the first and second widths W1, W2 may extend substantially parallel to the base 20. One skilled in the art will appreciate that the first and second widths W1, W2 can be measured in mutually perpendicular MD and CD directions if the head 50 as a whole is oriented substantially parallel to the base 20, FIGS. 3-5, 7, 8, 16. In some instances, wherein the head 50 is oriented at an angle relative to the base 20, the first and second widths W1, W2 can be measured along the head's major extensions cross-sectional projections of which are substantially parallel to the mutually perpendicular MD and CD directions, FIGS. 17, 18.

In the exemplary embodiment of FIG. 17, the projection 30 comprises the stem 40 that is substantially perpendicular to the base 20 and the head 50 that as a whole is inclined relative to the base 20. Consequently, the second width W2 can be measured in this embodiment along the major extension of the head 50, the projection of which in the cross-section (or the side view shown) is parallel to the CD direction. In the exemplary embodiment of FIG. 18, the projection 30 comprises the stem 40 that is inclined relative to the base 20 and the head 50 that as a whole is inclined relative to the base 20. The second width W2 can be measured in this embodiment along the major extension of the head 50, the projection of which in the cross-section (or the side view shown) is parallel to the CD direction. In the embodiment of FIG. 18, the width W2 can also be measured perpendicular to the third direction 23 of the stem's longitudinal axis L because the head 50 is oriented substantially perpendicular to the third direction 23 or the axis L. However, other embodiments are possible, in which both the stem 40 and the head 50 as a whole are inclined relative to the base 20—but in which the angle between the third direction 23 and the major extension of the head 50 in the relevant cross-section or side view is other than about 90 degrees. All of these embodiments are in the scope of this disclosure.

The thickness T can be substantially perpendicular to at least one of the first thickness W1 or the second thickness W2. In some embodiments, the thickness T may extend substantially parallel to the third direction 23. The thickness T of the head 50 can be measured as an overall thickness extending between the “highest” and “lowest” surfaces of the head 50, along the third direction 23 or the longitudinal axis L, FIG. 3.

The head 50 includes at least two cantilevered portions 60 outwardly extending from the stem 30, FIGS. 3, 4, 5, 7, and 8. The cantilevered portions 60 can generally extend in at least one of the first direction 21 and the second direction 22. Analogously to the head's orientation, for the cantilevered portion 60 to generally extend in the first direction 21 or the second direction 22, the cantilevered portion 60 does not need to be parallel to the first direction 21 or the second direction 22; it is sufficient that the cantilevered portion's cross-sectional projection taken in the plane of the first direction 21 be parallel to the first direction 21 or that the cantilevered portion's cross-sectional projection taken in the plane of the second direction 22 be parallel to the second direction 22. In some embodiments, the cantilevered portion or portions 60 can extend generally perpendicular to the third direction 23. In some embodiments, the cantilevered portion or portions 60 can extend generally parallel to the base 20.

The at least two cantilevered portions 60 can, but do not have to, be mutually opposite. The at least two cantilevered portions 60 can extend at an angle relative to one another. In further embodiments, an individual cantilevered portion 60 and/or at least two cantilevered portions 60 can extend at an acute or obtuse angle relative to the stem 40 or its longitudinal axis L. In still other embodiments, the individual cantilevered portion 60 and/or at least two cantilevered portions 60 can extend at an acute or obtuse angle relative to the base 20—or, alternatively, to extend substantially parallel to the base 20. All of these embodiments are in the scope of this disclosure.

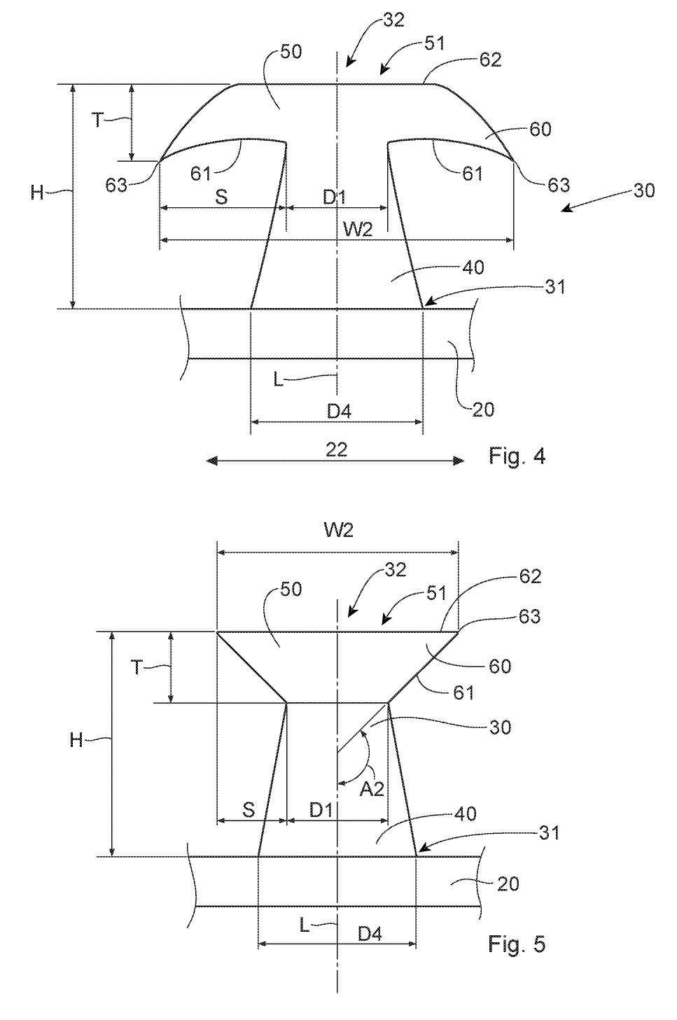

The individual cantilevered portion 60 has a bottom surface 61 facing the base 20 and a top surface 62 opposite to the bottom surface 61. In some embodiments, the bottom surface 61 and the top surface 62 can meet one another at an acute angle—to form, in combination with one another, an edge 63 disposed at a distance from the stem 40, FIGS. 4, 5, 8, 14. In some embodiments, the edge 63 can be relatively sharp—and can have ability to at least partially penetrate the texture of the papillae on the top surface of the tongue. In use, a relatively sharp surface of the edge 63 can beneficially have a “plow” effect when a user moves the tongue cleaner 10 in contact with the tongue's top surface, thereby facilitating removal of bacteria, food particles, and the breakdown products of foods that are trapped in the crevices between the tongue's papillae.

In some embodiments, the tongue cleaner 10 comprises a plurality of projections 30 in which a substantial portion of the bottom surface 61 of the cantilevered portions 60 can be inclined toward the stem 40, FIGS. 3, 4. This means that an included average angle A formed between a substantial portion of the bottom surface 61 and the longitudinal axis L of the stem 40 is less than 90 degrees, FIG. 3. More specifically, in the “inclined-toward” configuration the included average angle A can be less than 85 degrees, less than 80 degrees, less than 75 degrees, less than 70 degrees, less than 65 degrees, less than 60 degrees, less than 55 degrees, less than 50 degrees, less than 45 degrees, less than 40 degree, less than 35 degrees, and less than 30.

As used herein, “substantial portion” of the bottom surface 61 refers to a contiguous portion thereof that can be greater than 30%, greater than 40%, greater than 50%, greater than 60%, greater than 70%, and greater than 80% of the entire bottom surface 61, as measured in a relevant cross-section of the projection 60. In other words, the “substantial portion” of the bottom surface 61 can be defined in a cross-sectional view thereof, taken either in the plane of the first direction 21 or in the plane of the second direction 22—or in a relevant direction perpendicular to the third direction 23 or the longitudinal axis L.

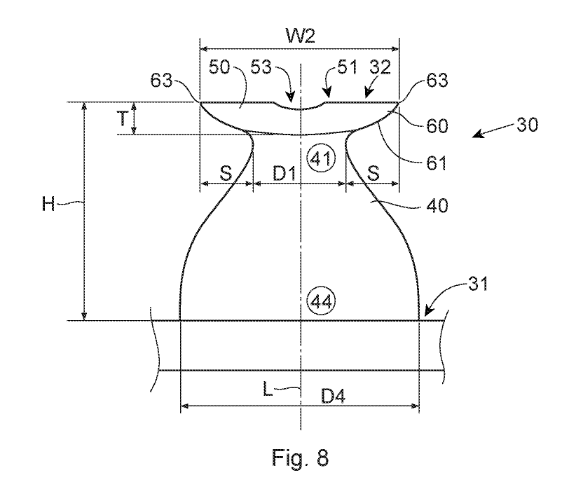

In other embodiments, the tongue cleaner 10 comprises a plurality of projections 30 in which a substantial portion of the bottom surface 61 of the cantilevered portions 60 can be inclined away from the stem 40, FIGS. 5, 8. This means that an included average angle A2 formed between a substantial portion of the bottom surface 61 and the axis L of the stem 40 is greater than 90 degrees, FIG. 5. More specifically, in the “inclined-away” configuration the included average angle A2 can be greater than 95 degrees, greater than 100 degrees, greater than 105 degrees, greater than 110 degrees, greater than 115 degrees, greater than 120 degrees, greater than 125 degrees, greater than 130 degrees, greater than 135 degrees, greater than 140 degree, greater than 145 degrees, and greater than 150 degrees.

Since the head 50, and hence its cantilevered portions 60, can extend in mutually perpendicular directions, an included angle existing in one direction (i.e., the first direction 21) between the stem's axis L and a substantial portion of the cantilevered portion's bottom surface 61 can be different from an included angle existing in the other direction (i.e., the second direction 22, which is perpendicular to the first direction 21) between the stem's axis L and a substantial portion of the cantilevered portion's bottom surface 61. Thus, in the exemplary embodiment of FIGS. 3 and 3A, an included average angle A existing in the second direction 22 between a substantial portion of the bottom surface 61 of the cantilevered portion 60 and the axis L is less than 90 degrees (FIG. 3) while an included average angle A1 existing in the first direction 21 between a substantial portion of the bottom surface 61 of the cantilevered portion 60 and the axis L is greater than 90 degrees (FIG. 3A).

In still other embodiments, the tongue cleaner 10 may comprise a plurality of first projections 30a and a plurality of second projections 30b, wherein in the plurality of first projections 30a a substantial portion of the bottom surface 61 of the cantilevered portions 60 can be inclined away from the stem 40, while in the plurality of second projections 30b a substantial portion of the bottom surface 61 of the cantilevered portions 60 can be inclined towards the stem 40, FIG. 2C.

In further embodiments, the tongue cleaner 10 can comprise a plurality of projections 30 having a first cantilevered portion 60a and a second cantilevered portion 60b, wherein a substantial portion of the bottom surface 61a of the first cantilevered portion 60a can be inclined towards the stem 40, while a substantial portion of the bottom surface 61b of the second cantilevered portion 60b can be inclined away from the stem 40, FIG. 14. Consequently, in an exemplary embodiment of FIG. 14, an included average angle A3 formed between the bottom surface 61a of the first cantilevered portion 60a and the longitudinal axis L of the stem 40 is acute, while an included average angle A4 formed between the bottom surface 61b of the second cantilevered portion 60b and the longitudinal axis L of the stem 40 is obtuse.

Other embodiments of the tongue cleaner 10 are also contemplated, in which the plurality of projections 30 may comprise any combination of the several embodiments described herein above. Although it is practically impossible to describe all of such embodiments, one skilled in the art will readily understand that any suitable arrangement of the embodiments described herein, variously combined, can be had in the tongue cleaner 10 of the disclosure—and all of them are included in the scope of this disclosure.

In some embodiments of the tongue cleaner 10, the bottom surface 61 of the cantilevered portion 60 can be at least partially curved. Such a curved or partially curved bottom surface 61 can have a shape selected from the group consisting of a convex shape (FIG. 14), a concave shape (FIGS. 3 and 4), and any combination thereof. The curved surfaces can be regularly or irregularly shaped. Portions of the curved surfaces that can form partially enclosed pockets 70 (FIGS. 7, 14) adjacent to a top section of the stem 40 could be particularly beneficial, because such partially enclosed pockets 70 could receive—and subsequently facilitate removal of—plaque substance formed on the surface of the tongue and containing bacterial microorganisms, extraneous cellular debris, and the like.

The included average angle formed between the longitudinal axis L of the stem 40 and a substantial portion of the bottom surface 61 can be computed using any conventional method for calculating an average parameter. For example, an average angle can be computed as an arithmetic average of a plurality of angles taken at equal intervals in the relevant cross-section (e.g., MD or CD) of the substantial portion of the bottom surface 61 of the cantilevered portion 60. If, e.g., the substantial portion of the bottom surface 61 includes a curved part or segment, then tangents taken at equal intervals on the curved segment can be used to compute the average angle.

Relevant dimensions of several exemplary embodiments of the individual projections 30 are shown in FIGS. 3, 3A, 4, 5, and 7. For example, the height H of the individual projection 30 can be from about 0.2 mm to about 0.6 mm. In one embodiment, the height H of the individual projection 30 is from about 0.39 mm to about 0.47 mm. The height H can be measured perpendicular to the base 20, between the first end 31 of the projection 30, i.e., the point of contact between the projection 30 and the base 20, and the second, free end 32 of the projection 30, i.e., the highest point thereof.

The first width W1 (FIG. 3A) of the head 50 of the individual projection 30 can be from about 0.1 mm to about 0.4 mm, and more specifically from about 0.2 mm to about 0.28 mm. The second width W2 (FIG. 3) of the head 50 of the individual projection 30 can be from about 0.2 mm to about 0.6 mm. In one particular embodiment, the second width W2 of the head 50 can be from about 0.34 mm to about 0.4 mm.

The thickness T of the head 50 of the individual projection 30 can be from about 0.02 mm to about 0.1 mm. In one particular embodiment, the thickness T of the head 50 is from about 0.03 mm to about 0.07 mm, and more specifically about 0.5 mm. The thickness T of the head 50 can be measured perpendicular to the major extension of the head 50. Thus, if the head 50 is oriented substantially perpendicular to the longitudinal axis L (as is shown, e.g., in FIGS. 3-5, 7, 8 and 18) then the thickness T of the head 50 can be measured parallel to the longitudinal axis L. If, on the other hand, the head 50 is angled relative to the longitudinal axis L (FIGS. 16 and 17), then the thickness T of the head 50 can be measured along a direction substantially perpendicular to the major extension of the head 50, or the width of the head 50, in the relevant cross-section or side view (the second width W2 in FIGS. 16 and 17). One skilled in the art will readily appreciate that if the head 50 is oriented substantially parallel to the base 20, that the thickness T of the head can be measured perpendicular to the base 20.

The individual cantilevered portion 60 has a length S extending in at least one of the first direction 21 and the second direction 22 and can be measured, in a relevant cross-section, from an uppermost or top surface of the stem's top section to the farthest free end of the cantilevered portion. The uppermost or top surface of the top section of the stem 10 is a portion of the stem's outer surface that is immediately adjacent to the bottom surface of the head 50, as viewed in a relevant cross-section. The length S can be from about 0.03 mm to about 0.2 mm, and more specifically from about 0.07 mm to about 0.13 mm.

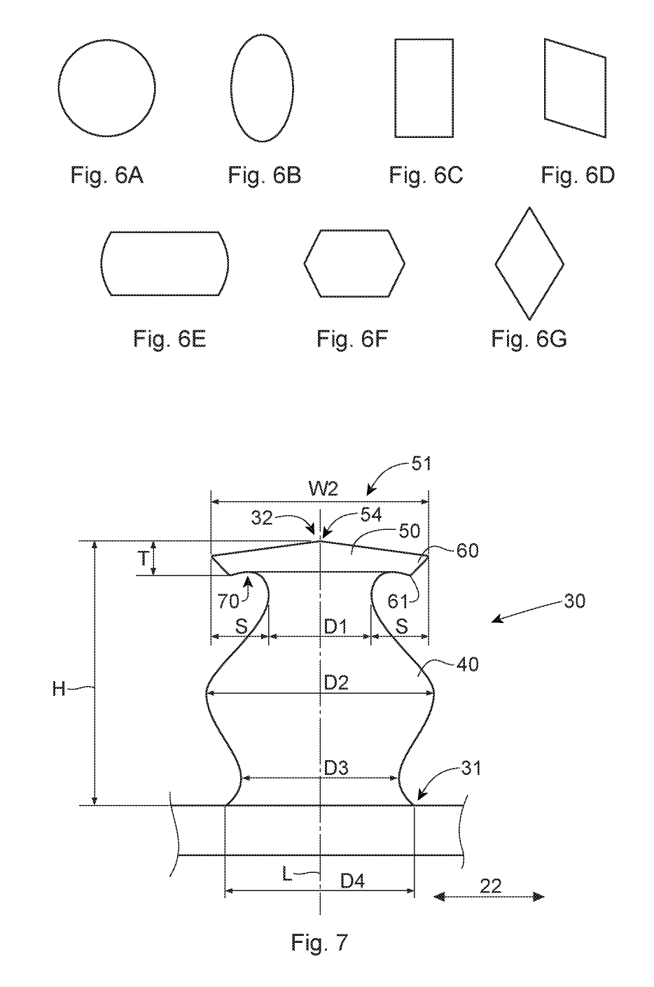

In plan view, the head 50 of the tongue cleaner 10, including its cantilevered portions 60, may comprise any suitable shape, e.g., a shape selected from a circular shape, a semi-circular shape, an elliptical shape, a polygon shape, a parallelogram shape, and any combination thereof. Several exemplary, non-limiting embodiments of such shapes are illustrated in FIGS. 6A-6G, wherein FIG. 6A schematically shows a top view of an exemplary embodiment of an individual projection comprising a head having a circular shape in plan view; FIG. 6B schematically shows a top view of an exemplary embodiment of an individual projection comprising a head having an oval or elliptical circular shape in plan view; FIG. 6C schematically shows a top view of an exemplary embodiment of an individual projection comprising a head having a rectangular shape in plan view; FIG. 6D schematically shows a top view of an exemplary embodiment of an individual projection comprising a head having a shape comprising parallelogram in plan view; FIG. 6E schematically shows a top view of an exemplary embodiment of an individual projection comprising a head having a shape comprising two mutually opposite straight sides and two mutually opposite curved sides; FIG. 6F schematically shows a top view of an exemplary embodiment of an individual projection comprising a head having a polygonal shape in plan view; and FIG. 6G schematically shows a top view of an exemplary embodiment of an individual projection comprising a head having a rhombus shape in plan view.

The plurality of projections 30 can be spaced from one another on the base 20 at a first pitch P1 extending parallel to the first direction 21 and a second pitch P2 extending parallel to the second direction 22. The pattern in which the projections 30 are spaced from one another on the base 20 may comprise an aligned arrangement (FIG. 2A), a staggered or alternating arrangement (FIG. 2B), or a mixed arrangement; the latter comprising both an aligned and staggered arrangements combined in any suitable configuration. Examples of bilaterally or unilaterally offset arrangements are shown in FIGS. 10, 11.

The first pitch P1 and the second pitch P2 can be substantially equal to one another. Alternatively, the first pitch P1 can be greater or smaller than the second pitch P2. In one embodiment, the first pitch P1 is greater than the second pitch P2. This embodiment may be particularly beneficial if the first direction 21 is parallel to the machine direction (MD) and the second direction 22 is parallel to the cross-machine direction (MD). In one particular embodiment, the first pitch P1 can be at least 50% greater than the second pitch P2. The first pitch P1 can be from about 0.8 mm to about 1.4 mm, and more specifically from about 0.9 mm to about 1.2 mm. The second pitch P2 can be from about 0.4 mm to about 0.7 mm, and more specifically from about 0.48 mm to about 0.62 mm.

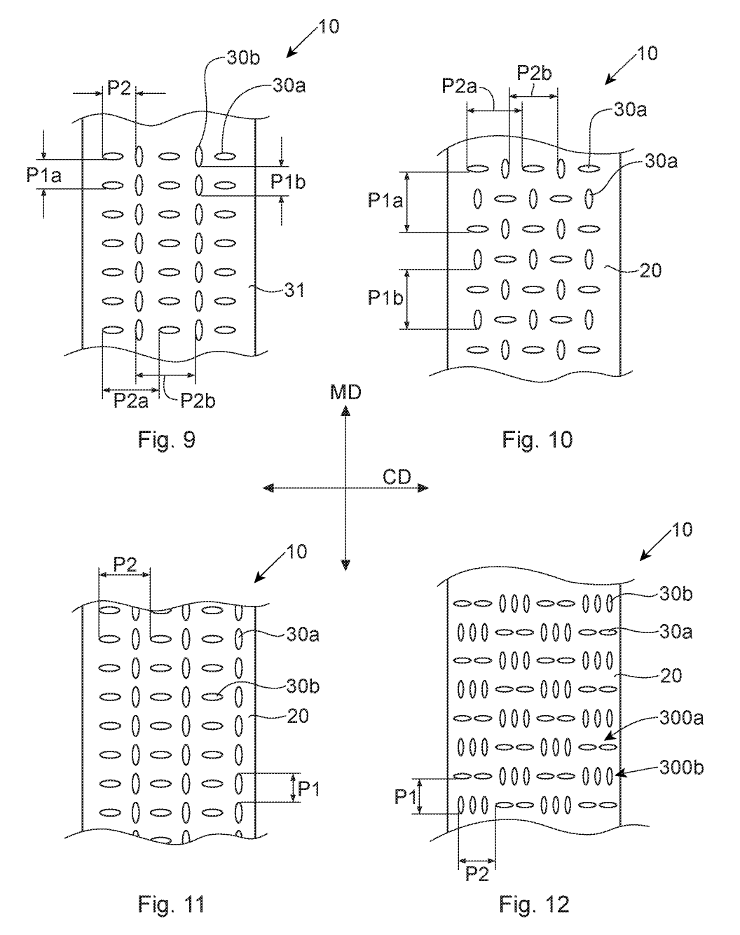

In an exemplary embodiment of FIG. 9, the pattern of projections comprises a plurality of first projections 30a and a plurality of second projections 30b different from the first projections 30a. The first projections 30a and the second projections 30b are arranged on the base to form uniform lines, each comprising either the first projections 30a or the projections 30b, wherein the lines formed by the first projections 30a alternate, in the second direction 22, with the lines formed by the second projections 30b. In such a configuration, a first pitch P1a and a second pitch P2a can be measured in the first direction 21 and the second direction 22, respectively, between adjacent first projections 30a. Likewise, a third pitch P1b and a fourth pitch P2b can be measured in the first and second directions 21, 22, respectively, between the second projections 30b. In addition, a pitch P2 can be measured, in the second direction 22, between adjacent first projection 30a and second projection 30b.

In some embodiments, the projections 30 can be spaced on the base 20 to form pluralities of clusters, or units. A single unit can comprise any number of projections, i.e., one, two, three, four, five, and more individual projections 30. In FIG. 12, the pattern of projections comprises a plurality of first units 300a and a plurality of second units 300b, bilaterally alternating in both the first and second directions 21, 22. In the exemplary embodiment of FIG. 12, each of the first units 300a comprises two first projections 30a, and each of the second units 300b comprises three second projections 30b. The pitch P1 in the first direction 21 and the pitch P2 in the second direction 22 can be measured between the first unit 300a and the second unit 300b, in both first and second directions 21, 22. The projections within the individual units can be spaced from one another at a pitch that is smaller than a corresponding pitch between the units.

The first projections 30a form a first density and the second projections 30b form a second density on the base 20. The first density and the second density can be equal or, alternatively, can differ from one another. At least one of the first density and the second density can be from about 60 projections per cm² to about 400 projections per cm² of the base, and more specifically from about 75 projections per cm² to about 350 projections per cm² of the base.

Moreover, in embodiments in which the projections 30 form plurality of units, or clusters, densities of individual clusters comprising the same type of projections can differ from one another. Thus, e.g., a hypothetical tongue cleaner (not shown) may comprise two clusters formed by the first projections 30b and three clusters formed by the second projections 30b, wherein the densities of the two first-projection clusters differ from one another and the densities of at least two of the three second-projection clusters differ from one another.

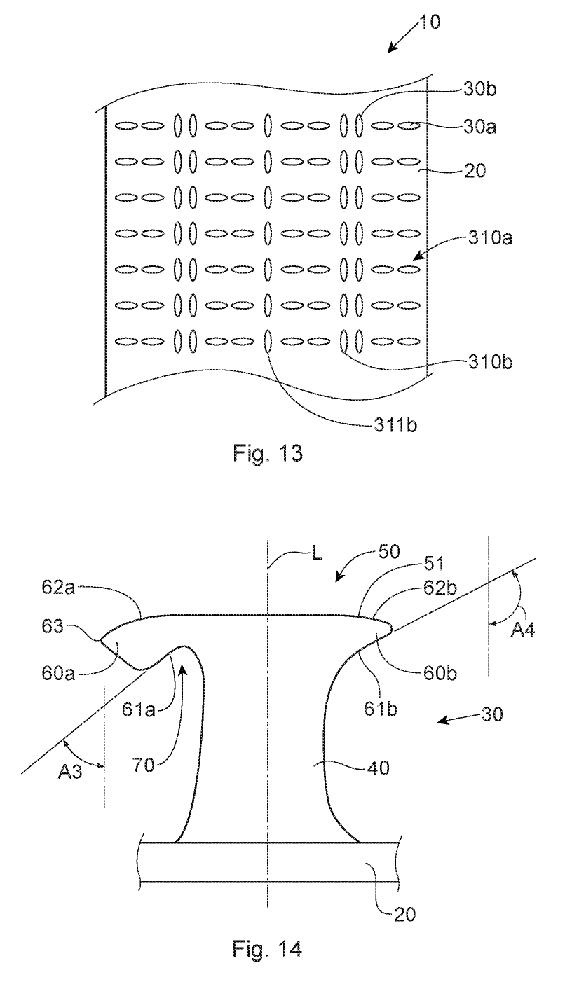

In an exemplary embodiment of FIG. 13, the pattern of projections comprises a plurality of first units 310a and a plurality of second units 310b, which are staggered in the second direction 22. Each of the first units 310a comprises two first projections 30a, and each of the second units 310b comprises two second projections 30b. There is also a plurality of third units 311b, each of the third units 311b comprising a single second projection 30b. One skilled in the art will appreciate that a great number of variations and combinations of suitable patterns of the projections 30 can be had—all of which are contemplated by the disclosure.

The stem 40 can have any suitable body. In one exemplary embodiment, shown in FIG. 3, the stem has a body comprising several sections extending along the longitudinal axis L and differing from one another by their relative thickness. While defining precise border lines separating the several sections having tapering portions from one another may be neither necessary nor practical in the present context, one skilled in the art will be able to readily distinguish between a top section 41 adjacent to the head 50, a bottom section 44 opposite to the top section 41 and adjacent to the base 20, and at least a first midsection 42 disposed between the top section 41 and the bottom section 44.

In the exemplary embodiment of FIG. 3, the top section 41 of the stem 40 can have a minimal thickness D1 of from about 0.15 mm to about 0.19 mm, as measured at the narrowest portion of the top section 41 in a direction substantially perpendicular to the third direction 23—or in some embodiments in at least one of the first and second directions 21, 22. The first midsection 42 can have a maximal thickness D2 measured at the widest portion of the first midsection 42 in a direction substantially perpendicular to the third direction. The thickness D2 of the first midsection 42 can be from about 10% to about 50% greater than the minimal thickness D1 of the top section 41. In the embodiment of FIG. 3, the stem 40 has a second midsection 43 intermediate the bottom section 44 and the first midsection 42. The second midsection 43 can have a minimal thickness D3 measured at the narrowest portion of the second midsection 43 in a direction substantially perpendicular to the third direction. The thickness D3 of the second midsection 43 can be equal or approximately equal to the minimal thickness D1 of the top section 41. The bottom section 44 of the stem 40 can have a thickness D4 measured at the widest portion of the first bottom section 44. In FIG. 3, the thickness D4 is the thickness of the stem 40 at the base 20. The thickness D4 of the bottom section 44 can be, e.g., from about 0.16 mm to about 0.22 mm.

In some embodiments of the tongue cleaner 10, the head 50 can be wider than the bottom section 44 of the stem 40, FIGS. 3-5. In other embodiments, the midsection 42 of the stem 40 can be wider than at least one of the head 50 and the bottom portion 44. In an exemplary embodiment of FIG. 7, the midsection 42 of the stem 40, having a thickness D2, is wider, in at least one of the first and second directions 21, 22, than the head 50, having a thickness W2, and the bottom portion 44, having a thickness D4, in their respective corresponding dimensions, along the second direction 22. In still other embodiments, the bottom section 44 of the stem 40 can be wider than the head 50, FIG. 8.

The head 50 can have any suitable top surface 51. The head's top surface 51 can be, e.g., substantially flat, FIG. 5. The top surface 51 can also, or alternatively, include convex or concave portions. For example, each of the exemplary embodiments of the projection 30 shown in FIGS. 3 and 8, has the top portion 51 of the head 50 comprising a centrally located depression 53. At the same time, the projection 30 shown in the exemplary embodiment of FIG. 7, has the head 50 comprising the top portion 51 that includes a centrally located apex 54.

FIGS. 15 and 16 show an exemplary embodiment of the tongue cleaner 10 in which projections 30 are inclined relative to the base 20. The projection 30 can be inclined to form an angle of about (90±30) degrees between the longitudinal axis L and the base 20. In other words, an angle formed between the longitudinal axis L of the individual projection 30 and the base 20 can be from about 60 degrees to about 120 degrees. In the exemplary embodiments of FIGS. 15 and 16 the individual projections 30 are inclined in the CD direction, or the second direction 22, but one skilled in the art would understand that the projections 30 can be inclined in the MD direction, or the first direction 21—or in any direction within 360 degrees between the CD and MD directions.

Also, in the exemplary embodiment of FIG. 15 the rows of projections 30 are shown as inclined, oppositely with respect to any two immediately adjacent rows, in the second direction 22 while unilaterally alternating in the first direction 21. But one skilled in the art would appreciate that a reverse embodiment can be had as well, in which the rows of projections 30 are inclined in the first direction 21 and alternate in the second direction 22. In further embodiments (not shown), the plurality of projections 30 can be inclined variously in two or more directions, e.g., the projections can be bilaterally staggered and/or bilaterally alternating, comprising regular and/or irregular overall patterns of projections on the base.

Moreover, embodiments are contemplated in which some of the projections 30 are inclined (uniformly, regularly, in series, or otherwise) relative to the base 20 while others are not; as well as embodiments in which the projections 30 differ from one another with respect to their heights H, first and second widths W1, W2 of the head 50, orientation, shape, and size of the cantilevered portions 60, and other relevant parameters described herein.

The tongue cleaner 10 of the disclosure can be made from any suitable polymeric material, such as, e.g., thermoplastic resins comprising polyolefins (polypropylene and polyethylene) polyvinyl chloride, polystyrene, nylons, polyester, such as, e.g., polyethylene terephthalate and the like, and copolymers and blends thereof. The polymeric resin can comprise a polypropylene, polyethylene, polypropylene-polyethylene copolymer, or blends thereof. The tongue cleaner 10 can be manufactured by any suitable method known in the art, e.g., by extruding a strip of thermoplastic resin from an extruder through a die. Optionally, the strip of thermoplastic resin can be stretched prior to cutting to desired shape of the tongue cleaner 10, to provide the resin with a beneficial molecular orientation. One of such processes is described in U.S. Pat. No. 7,185,401, the disclosure of which is incorporated herein by reference for the purpose of describing processes that can be utilized for making the tongue cleaner, as well as materials and equipment suitable therefor.

While particular embodiments have been illustrated and described herein, various other changes and modifications may be made without departing from the spirit and scope of the invention. It is to be understood, therefore, that the scope of the claims is not limited to the specific embodiments described and shown herein, and that the terminology used herein for the purpose of describing particular embodiments by way of example only is not intended to be limiting to the embodiments shown. Moreover, although various aspects of the invention have been described herein, such aspects need not be utilized in combination. It is therefore intended to cover in the appended claims all such changes and modifications that are within the scope of the invention.

The terms “substantially,” “essentially,” “about,” “approximately,” and the like, as may be used herein, represent the inherent degree of uncertainty that may be attributed to any quantitative comparison, value, measurement, or other representation. These terms also represent the degree by which a quantitative representation may vary from a stated reference without resulting in a change in the basic function of the subject matter at issue. For example, as used herein, the terms “substantially perpendicular,” “substantially parallel,” and the like indicate general—as opposed to strict—orientation of an element being described. In other words, an element that is substantially parallel (or perpendicular) to a certain direction or surface does not need to be strictly parallel (or perpendicular) to that direction or surface. All that is required is that the element extends substantially in a direction that is approximately parallel (or perpendicular) to the referenced direction or surface.

Also, the term “direction” is used herein to describe a relative orientation (e.g., as within the Cartesian coordinate system)—rather than a vector. Thus, phrases such as “an element extending in a first direction,” “an element extending substantially parallel to the first direction,” and the like, mean that the element being described is substantially parallel to the first direction, wherein some degree of deviation from the first direction is acceptable.

Further, the dimensions and values disclosed herein are not to be understood as being strictly limited to the exact numerical values recited. Instead, unless otherwise specified, each such dimension is intended to mean both the recited value and a functionally equivalent range surrounding that value. For example, values disclosed as “0.2 mm” or “30 degrees” are intended to mean, respectively, “about 0.2 mm” or “about 30 degrees.” Also, as used in the specification, including the claims, the singular forms “a,” “an,” and “the” include the plural, and the phrase “at least one” includes one or several. Further, any of the “open” terms including words “comprising,” “having,” “containing,” and “including” means that other elements, steps, ingredients, et cetera, that do not adversely affect the relevant end result, can be added. Each of these terms encompasses, but is not limited by, the terms “consisting of” and “consisting essentially of.”

The disclosure of every document cited herein, including any cross-referenced or related patent application or patent, and any patent application or patent to which this application claims priority or benefit thereof, is hereby incorporated herein by reference unless expressly excluded or otherwise limited. The citation of any document is not an admission that it is prior art with respect to any invention disclosed or claimed herein—or that it alone, or in any combination with any other reference or references, teaches, suggests, or discloses any such invention. Lastly, to the extent that any meaning or definition of a term in this document conflicts with any meaning or definition of the same or similar term in a document incorporated by reference, the meaning or definition assigned to or contextually implied by that term in this document shall govern.

What is claimed is:

1. A tongue cleaner comprising:
a base having an first direction and a second direction perpendicular to the first direction and a plurality of projections outwardly extending from the base in a third direction;
wherein at least some of the projections have a first end attached to the base, a second free end opposite to the first end, and a length extending between the first end and the second end;
wherein the at least some of the projections comprise an elongated stem and a head integral with the stem and disposed at a distance from the base, the stem having a longitudinal axis oriented substantially parallel to the third direction;
wherein the head comprises at least one cantilevered portion outwardly extending from the stem and having a bottom surface generally facing the base and a top surface opposite to the bottom surface;
wherein the plurality of projections comprises at least first projections and second projections;
wherein a substantial portion of the bottom surface of the individual first projections is inclined away from the stem; and
wherein a substantial portion of the bottom surface of the individual second projections is inclined towards the stem.

2. The tongue cleaner of claim 1, wherein the pattern comprises a plurality of first units and a plurality of second units, wherein each of the first units comprises the first projections and each of the second units comprises the second projections, wherein the plurality of first units comprises a plurality of first rows extending in at least one of the first direction and the second direction and the plurality of second units comprises a plurality of second rows extending in at least one of the first direction and the second direction.

3. The tongue cleaner of claim 2, wherein at least one of the first units comprises a plurality of first projections.

4. The tongue cleaner of claim 3, wherein the first projections in the at least one of the first units are spaced from one another at a pitch of from about 0.4 mm to about 1.4 mm in at least one of the first and second directions.

5. The tongue cleaner of claim 2, wherein at least one of the second units comprises a plurality of second projections.

6. The tongue cleaner of claim 5, wherein the second projections in the at least one of the second units are spaced from one another at a pitch of from about 0.4 mm to about 0.7 mm in at least one of the first and second directions.

7. The tongue cleaner of claim 2, wherein the first units alternate with the second units in at least one of the first direction and the second direction.

8. The tongue cleaner of claim 2, wherein the first units are unilaterally offset relative to the second units in one of the first and second directions.

9. The tongue cleaner of claim 2, wherein the first units are bilaterally offset relative to the second units in both the first and second directions.

10. The tongue cleaner of claim 2, wherein at least some of the first units are equidistant from the second units immediately adjacent thereto, and wherein at least some of the second units are equidistant from the first units immediately adjacent thereto.

11. The tongue cleaner of claim 1, wherein the first projections form a first density and the second projections form a second density.

12. The tongue cleaner of claim 11, wherein at least one of the first density and the second density is from about 60 projections per cm² to about 400 projections per cm² of the base.

13. The tongue cleaner of claim 11, wherein the first density is different from the second density.

14. The tongue cleaner of claim 1, wherein the projections are spaced from one another to form a pattern having a first pitch extending parallel to the first direction and a second pitch extending parallel to the second direction, wherein the first pitch is greater than the second pitch.

15. The tongue cleaner of claim 14, wherein the first pitch is at least 50% greater than the second pitch.

16. The tongue cleaner of claim 14, wherein the first pitch is from about 0.4 mm to about 1.4 mm and the second pitch is from about 0.4 mm to about 0.7 mm.

17. The tongue cleaner of claim 14, wherein the first pitch is from about 0.8 mm to about 1.3 mm and the second pitch is from about 0.48 mm to about 0.62 mm.

18. The tongue cleaner of claim 1, wherein a height of at least some of the projections is from about 0.2 mm to about 0.55 mm.

19. The tongue cleaner of claim 1, wherein the head of the projection comprises, in plan view, a shape selected from a circular shape, a semi-circular shape, an elliptical shape, a polygon shape, a parallelogram shape, and any combination thereof.

20. The tongue cleaner of claim 1, wherein the head has a thickness from about 0.03 mm to about 0.07 mm.

21. The tongue cleaner of claim 1, wherein the first projections and the second projections in combination form a pattern having a combined density of from about 60 projections per cm² to about 400 projections per cm² of the base.

22. An oral-care implement comprising the tongue cleaner of claim 1, wherein the oral-care implement is selected from the group consisting of manual toothbrush, electric toothbrush, a unitary tongue-cleaning device, and any combination thereof.
